(19) United States
(12) Patent Application Publication
(10) Pub. No.: US 2025/0237721 A1
(43) Pub. Date: Jul. 24, 2025

(54) SYSTEMS AND METHODS FOR DETERMINING POSITION OF METAL OBJECT IN DISTORTED MRI

(71) Applicants: Case Western Reserve University, Cleveland, OH (US); Vanderbilt University, Nashville, TN (US)

(72) Inventors: William A. Grissom, Cleveland, OH (US); Sarah Garrow, Cleveland, OH (US); Daniel Esser, Nashville, TN (US)

(21) Appl. No.: 19/025,262

(22) Filed: Jan. 16, 2025

Related U.S. Application Data
(60) Provisional application No. 63/623,688, filed on Jan. 22, 2024.

(51) Int. Cl. G01R 33/28; A61B 5/055; A61B 17/34; G01R 33/48; G01R 33/56

(52) U.S. Cl. CPC G01R 33/286; A61B 5/055; A61B 17/3403; G01R 33/283; G01R 33/4818; G01R 33/5608

(57) ABSTRACT

A method for generating a magnetic resonance (MR) image in the presence of an interventional device that induces distortions is provided. The method includes using a processor to perform steps that include accessing a distorted MR image of a subject with the interventional device arranged in the subject. The steps also include using the distorted MR image to determine parameters of a set of spatially differentiable functions that describe an estimated position of the interventional device in the subject. The steps further include determining a magnetic susceptibility map based on susceptibility properties of the interventional device and the estimated position of the interventional device. The steps also include generating a simulated MR image by modifying a reference image of the subject based on the magnetic susceptibility map and updating the estimated position using the simulated MR image to generate an updated position of the interventional device in the subject.

### CROSS-REFERENCE TO RELATED APPLICATIONS

This application claims priority under 35 U.S.C. § 119 to U.S. Provisional Patent Application No. 63/623,688 filed on Jan. 22, 2024. The entire contents of which is incorporated herein by reference.

### STATEMENT REGARDING FEDERALLY SPONSORED RESEARCH

This invention was made with government support under NS120518 awarded by the National Institutes of Health. The government has certain rights in the invention.

### BACKGROUND

Magnetic resonance imaging (MRI) provides valuable real-time guidance for many surgical interventions including biopsies, neurosurgeries, spinal interventions, ablation therapies (e.g., microwave, cryogenic, radiofrequency, laser), joint injections, cardiovascular catheter procedures, drainage procedures, focused ultrasound therapies, deep brain stimulation, and others. Many of these require the use of metallic needles or other devices made of stainless steel, titanium, or nitinol, which are not ferrous but nevertheless distort the scanner's main magnetic field (B₀) due to their very different magnetic susceptibility compared to soft tissue. The distortions in the magnetic field lead to resonance frequency distortions that cause imaged signals to be spatially mis-registered in the reconstructed images. This occurs in both the slice dimension of a slice-selective imaging scan, and in the frequency encoded dimension of any scan. The strong spatial gradients of the frequency distortions also lead to a region of signal loss around a needle that can be several millimeters or more in width. The width of these signal-free regions and the image distortions limit the precision with which the device's position and pose or orientation can be determined.

Several imaging approaches have been developed to minimize image distortion around needles or other implanted metals in the body. View angle tilting compensates slice-dimension distortions but induces in-plane blurring. One of the more distortion-robust imaging methods is to apply a wideband excitation of the entire 3D volume and then apply only phase encoding, but this requires hours of imaging which is incompatible with an intervention, and it does not address signal loss around the needle. Multi-spectral imaging methods make a tradeoff by adding a small amount of phase encoding in the slice dimension to resolve slice distortions around a needle, with view angle tilting to mitigate frequency encoding distortions. These methods still require long scan times of several minutes or more which limits their intraprocedural use, and only modest reductions in needle artifact size have been reported to date. The “white marker” scan uses additional gradient pulses to spoil signal distant from a needle while refocusing signal near the needle, making it appear bright in an otherwise dark image. This can then enable identification of the needle location using e.g., principal component analysis (PCA) of the bright voxels' coordinates. However, white marker imaging requires tuning of the gradients depending on several factors, including the specific needle's magnetic susceptibility and its orientation with respect the main field. Needles themselves can also be modified to reduce magnetic field distortions. For example, needle material composition can be modified, but this usually requires a tradeoff with stiffness. As another example, active shielding can be added to the needle, but this typically requires the introduction of potentially dangerous electric currents and increases the needle size, and the currents have to be continuously adjusted as the needle moves or changes orientation.

Thus, no existing approach has been able to eliminate or suitably control the image distortions caused by needles in the context of a broad range of clinical purposes. Most approaches require major tradeoffs in scan time while only partially addressing distortion, or additional instrumentation. Accordingly, there is an ongoing need for improved methods and systems for determining the position of metal objects, such as needles, in MRI.

### SUMMARY OF THE DISCLOSURE

The present disclosure addresses the aforementioned drawbacks by providing a system and method for estimating the position of an interventional device in a distorted image or producing a magnetic resonance (MR) image in the presence of an interventional device.

In some aspects, a method for generating a magnetic resonance (MR) image in the presence of an interventional device that induces distortions is provided. The method includes using a processor to perform steps that include accessing a distorted MR image of a subject with the interventional device arranged in the subject. The steps also include using the distorted MR image to determine parameters of a set of spatially differentiable functions that describe an estimated position of the interventional device in the subject. The steps further include determining a magnetic susceptibility map based on susceptibility properties of the interventional device and the estimated position of the interventional device. The steps also include generating a simulated MR image by modifying a reference image of the subject based on the magnetic susceptibility map and updating the estimated position using the simulated MR image to generate an updated position of the interventional device in the subject.

In other aspects, a method for determining a position of an interventional device arranged in a subject during a magnetic resonance imaging (MRI) acquisition is provided. The method includes using a processor to perform steps that include accessing a reference image that represents an undistorted MR image of a subject without the interventional device arranged in the subject. The steps also include accessing a distorted MR image of the subject with the interventional device arranged in the subject acquired using a pulse sequence and accessing parameters of the pulse sequence. The steps also include estimating a position of the interventional device based on the distorted MR image of the subject. The steps further include determining a magnetic field distortion map based on a magnetic susceptibility distribution of the interventional device and the estimated position of the interventional device. The steps also include modifying the reference image based on the magnetic field distortion map to produce a simulated image. The steps further include updating the estimated position of the interventional device by comparing the simulated image with the distorted MR image to determine a position of the interventional device arranged in the subject.

In still other aspects, a system generating a magnetic resonance (MR) image in the presence of an interventional device that induces distortions is provided. The system includes a processor that is configured to access a distorted MR image of a subject with the interventional device arranged in the subject. The processor is also configured to use the distorted MR image to determine parameters of a set of spatially differentiable functions describing an estimated position of the interventional device in the subject. The processor is also configured to determine a magnetic susceptibility map based on susceptibility properties of the interventional device and the estimated position of the interventional device. The processor is also configured to generate a simulated MR image by modifying a reference image of the subject based on the magnetic susceptibility map and update the estimated position using the simulated MR image to generate an updated position of the interventional device in the subject.

In other aspects, a method for determining a position of an interventional device arranged in a subject during an MRI acquisition is provided. The method includes using a processor to access a distorted MR field map measured while the interventional device is arranged in the subject acquired using a pulse sequence. The method also includes using the processor to estimate a position of the interventional device based on the distorted MR field map of the subject and simulate a magnetic field distortion map based on a magnetic susceptibility distribution of the interventional device and the estimated position of the interventional device. The method further includes using the processor to update the estimated position of the interventional device by comparing the simulated magnetic field distortion map with the distorted MR field map to determine a position of the interventional device arranged in the subject.

These are but a few, non-limiting examples of aspects of the present disclosures. Other features, aspects and implementation details will be described hereinafter.

### BRIEF DESCRIPTION OF THE DRAWINGS

Various objects, features, and advantages of the disclosed subject matter can be more fully appreciated with reference to the following detailed description of the disclosed subject matter when considered in connection with the following drawings, in which like reference numerals identify like elements.

FIG. 1A provides a flowchart setting forth steps of an example process for estimating the position of an interventional device in a distorted magnetic resonance image.

FIG. 1B provides a flowchart setting forth steps of another example process for estimating the position of an interventional device in a distorted magnetic resonance field map.

FIG. 2 provides a flowchart setting forth steps of another process for estimating the position of an interventional device in a distorted magnetic resonance image.

FIG. 3 shows simulated results in an example implementation of the methods described in the present disclosure.

FIG. 4A demonstrates a simulated curved needle and resulting image distortion and incorrect position estimation, which can be improved using the systems and methods described in the present disclosure.

FIG. 4B demonstrates the use of curved needles for interventional procedures in accordance with the present disclosure.

FIG. 5A shows the differentiable Gaussian and analytical needle susceptibility models.

FIG. 5B shows the distortion calculated based on the differentiable Gaussian and analytical needle models.

FIG. 6 illustrates an algorithm that can be used to recover needle position from measured images in accordance with the present disclosure.

FIG. 7 shows simulation data for the needle localization on the cross section of a shifted and rotated needle.

FIG. 8A shows example simulation results for a needle in various orientations generated using methods described herein.

FIG. 8B shows additional example simulation results for pulse sequences with various bandwidths generated using methods described herein.

FIG. 9 is a block diagram of an example magnetic resonance imaging (“MRI”) system that can implement the methods described in the present disclosure.

FIG. 10 is a block diagram of an example interventional imaging system that can implement the methods of the present disclosure.

FIG. 11 is a block diagram of example components that can implement the system of FIG. 10.

### DETAILED DESCRIPTION

Before any aspects of the present disclosure are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The following discussion is presented to enable a person skilled in the art to make and use embodiments of the invention. Various modifications to the illustrated embodiments will be readily apparent to those skilled in the art, and the generic principles herein can be applied to other embodiments and applications without departing from embodiments of the invention. Thus, embodiments of the invention are not intended to be limited to embodiments shown but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of embodiments of the invention. Skilled artisans will recognize the examples provided herein have many useful alternatives and fall within the scope of embodiments of the invention.

Magnetic resonance (MR)-guided needle-based and other interventions require accurate and precise determination of the needle position to display to the surgeon. Pulse sequences and processing methods have been proposed to determine needle positions in real time, but the magnetic susceptibility of metal needles induces distortions that produce errors in these estimates. Such distortions may include spatial distortions (e.g., a warping in space) that can occur in plane (e.g., in the frequency encoded direction) and spatial distortion of the slice selection or excitation profile. The distortions may also include signal dropout or signal loss caused by intravoxel dephasing.

The distortions and subsequently-estimated needle positions depend on the orientation of the needle with respect to B₀, as well as sequence parameters, such as pixel bandwidth. Further, curved needles are the best choice for some procedures, such as transforamenal hippocampal ablation to treat epilepsy. However, current image-based needle localization methods do not apply to curved needles, and further cannot account for possible needle flexing. Moreover, curved or deformable needles can produce more complex distortions, including increased pile-up effects, due to the complex susceptibility profile resulting from their curved shapes.

Thus, the present disclosure addresses the aforementioned drawbacks by providing systems and methods for determining the position of a needle or other object in distorted MR images. While the systems and methods are primarily described herein in the context of MRI in the presence of metal needles, it is understood that the described mechanisms can be applied in the context of any external object that produces susceptibility-induced distortions. For example, the object may be an interventional object that is made of metal. The object may also be made or include another material that has a susceptibility that differs from soft or other biological tissue. The interventional object may include a needle, a biopsy clip, a deep brain stimulation lead, an electrode lead, an ultrasound transducer, a stimulation device, a fiducial marker, a pacemaker, an implant (e.g., hip implant), or another device implanted into a patient's body.

In some aspects, a differentiable needle and image distortion model are presented that address previously unmet needs by enabling recovery of needle position and orientation directly from a measured image using an optimization technique (e.g., a gradient-based optimizer). Such a model inherently leverages image distortions that typically produce positioning errors, to instead more accurately characterize the needle position. The model can additionally enable determination of needle flexing. In some implementations, needle susceptibility maps can be modeled using a collection of differentiable functions, such as Gaussian functions, which are related differentiably to a simulated distorted image. This simulated image can be iteratively compared to a measured image to determine the true needle position in space.

The present disclosure recognizes that the magnetic field distortions can be calculated due to a metallic needle if the needle's magnetic susceptibility and shape are known, and it is possible to calculate MR image distortions based on those magnetic field distortions. Furthermore, in some implementations, these relationships are differentiable and hence amenable to numerical optimization. With this recognition, the present disclosure provides systems and methods to precisely and accurately determine the position and orientation of a needle in tissue in which a simulated image is compared to the actual image, and the needle position (e.g., translation, rotation, and deformation) is updated to make the simulated and actual images match. The systems and methods can be applied to any MRI scan type.

To this end, systems and methods are provided in which an MRI image of a needle in tissue is acquired. The needle's magnetic susceptibility distribution is then used to calculate a magnetic field distortion. The magnetic field distortion is used to calculate an MRI image or a distortion map that is multiplied into or otherwise used to modify an undistorted MRI image to obtain a simulated MR image. The needle's modeled position is adjusted to make the acquired and simulated images match. The needle can be represented by a function or a set of functions that are differentiable with respect to their spatial positions and orientations, such as a set of Gaussian functions.

The needle's position can be iteratively updated until a match is obtained. In some implementations, the needle's position can be determined using a gradient-based optimization. In some implementations, a dictionary of possible distorted images or image distortions can be pre-calculated, and the best match can be selected from the dictionary, yielding the needle's position and orientation. In still other implementations, this process can combine an iterative method to determine one of the needle's translational position and orientation, and a dictionary method can be used to determine the other of the needle's translational position and orientation.

A dipole field convolution method can be used to calculate the frequency or field distortions from the needle's susceptibility distribution. A Bloch equation simulation of the pulse sequence can be used to calculate the image. A fitting can be performed directly in the Fourier domain using a distortion image multiplied into a pre-acquired image without the needle present. The fitting can be applied to multiple acquired images with different contrasts simultaneously. The distortions in excited, inverted, saturated, or refocused profiles can be included in the distorted image calculation. The distortions in the frequency encoded dimension of the image can be included in the distorted image calculation. The signal loss due to intravoxel phase dispersion can be included in the distorted image calculation.

The needle's position can be determined by calculating the derivatives of an objective function measuring the error between the acquired and simulated MR images and using those derivatives to reduce the value of the objective function until the images match or nearly match. An autodifferentiation can be used to calculate the derivatives. The needle's position can be initialized from previous known positions or based on simple image metrics such as the center of mass of the signal void produced by the needle in the MR image or principal component analysis (PCA). A regularization can be used to discourage the needle functions from deviating from the needle's unbent shape or deviating too much from a previously known position. A set of needle function coordinates can be allowed to shift with respect to each other at select locations along the needle, which may be based on known bending properties of the needle.

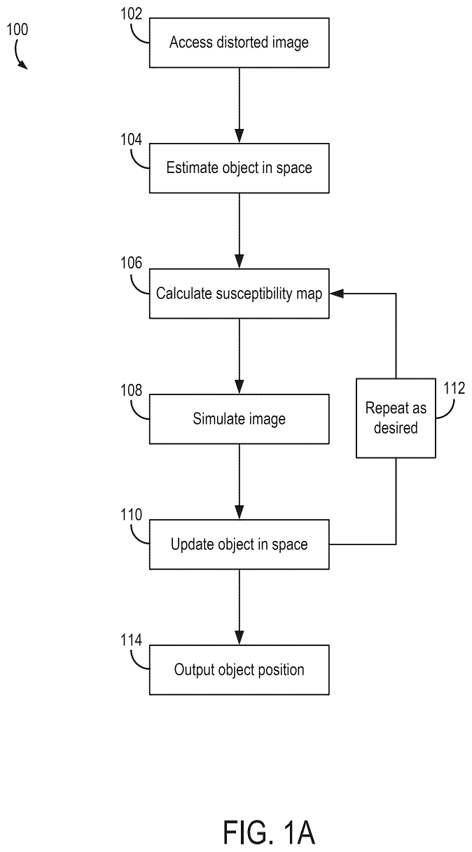

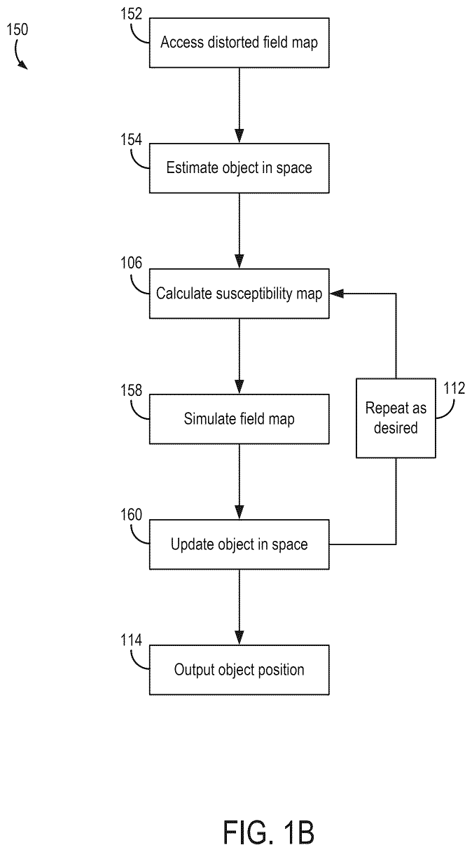

Referring now to FIG. 1A, a process 100 is presented that can be used to generate an MR image in the presence of an object that induces distortions. For example, the object may be arranged in a subject or patient. The object may have a susceptibility different than that of biological tissue such that it causes distortions in MR images. Such object may be an interventional device. For example, the device may include a needle, a biopsy clip, a deep brain stimulation lead, an electrode lead, a fiducial marker, a pacemaker, or an implant (e.g., orthopedic implant).

The process 100 can be performed by a processor or other computer system. The processor can be in communication with a data source or MRI system. In some implementations, the processor can also be in communication with a robotic system that can control robotic movement of the interventional device. In some implementations, the processor can be in communication with a user interface, which may include a user display to show the position of the interventional device.

The process 100 can be used to estimate the position of the interventional device based, in part, on an MR image acquired in the presence of the device. This position may include a translational position (e.g., in x, y, and z), which may be referred to as position, linear position, location, coordinates, displacement, or shift. The position may also include an orientation of the device, which may be referred to as rotation, angle, or angle of rotation. The position may also include deformation of the device, which may be referred to as flex, flexing, bending, deformation, or shape.

The position of the device may be described by parameters that characterize the position of a model representation of the device. For example, these parameters may include a mean shift of the model (e.g., position of the model center) that can be described by cartesian coordinates. The parameters may also include a rotation matrix that describes the orientation of the model. In some implementations, the model representation may be adjusted in use to allow for deformation of the device. Thus, the model representation may include variables (e.g., individual shifts) that describe parts of the model representation (e.g., a shift of each Gaussian function, as will be described).

Process 100 will be described in the context of the imaging domain. However, it should be understood that such process can be similarly performed in the frequency domain or k-space using corresponding methods, so that no image reconstruction of the acquired data is performed. Moreover, in some cases, performing the optimization in the k-space domain may be advantageous. For example, undersampled k-space data can be used as acquired (e.g., without filling in unacquired data). Such use of k-space data may advantageously circumvent the discontinuous image-domain representation of the device caused by aliasing. As another example, performing in the optimization in the k-space domain could enable mixing of data between consecutive image acquisitions in a flexible way. For example, a sliding window can be used to select the data used for optimization. This may allow for reduced computational load and faster optimization updates.

Process 100 includes accessing an MR image, as in process block 102. For example, the image may be acquired from a subject while the interventional device is arranged in the subject. Such acquired image may be distorted due to the presence of the object, and therefore may be referred to as a distorted MR image. The acquired MR image is associated with pulse sequence parameters, such as pixel bandwidth and echo time, which describe the pulse sequence used to acquire the image. In some implementations, the distorted image may be acquired with a resolution high enough to sufficiently image the interventional device (e.g., 2 mm×2 mm or higher in plane resolution for a needle). In some implementations, the imaging data accessed may have been acquired with undersampling in the k-space domain.

In some implementations, process block 102 may include accessing pulse sequence parameters that are associated with the pulse sequence used to acquire the distorted image. Process block 102 may also include accessing or measuring a distorted magnetic field (B₀) map that is associated with the distorted MR image. Such map characterizes the main magnetic field (B₀) in the presence of the interventional device.

Process block 102 can also include accessing a reference image that is associated with the distorted image. Such reference image can represent the anatomy or same imaging volume of the distorted image. However, the reference image represents an undistorted MR image without the interventional device arranged in the volume. In this way, the reference image can be measured (e.g., by acquiring an image prior to the introduction of the device) or estimated (e.g., by modifying the distorted MR image). The reference image may be a real-valued or complex-valued image.

In some implementations, process block 102 can include acquiring images using white marker tracking methods in which the background signal is dephased using a gradient while the signal near the device is preserved as the dipole field induced by the device compensates for the gradient-induced dephasing. Such data can be used to produce an estimate of the device position. These white marker tracking methods may also be used to produce a reference image.

The process 100 also includes generating an initial estimate of the object's position in space, as in process block 104. Such position can be estimated based on a distorted image accessed in process block 102 or based on a previously known position of the device.

In some implementations, the position can be estimated by masking signal dropout in the image, which is typically caused by the interventional device. For example, a mask can be produced by thresholding the image signal. A mask can also be produced by manual input from a user (e.g., drawing an estimated position). The position can be estimated by calculating a center of mass of the mask. For example, the center of mass can be determined using a spatial mean of the mask or a weighted mean of the signal dropout region. In some implementations, the position can be estimated using a PCA approach by performing a PCA of the mask.

In some implementations, the position can be estimated using a dictionary. For example, a dictionary can be formed by simulating a set of images in which the device is arranged in various positions. For example, these simulated images can be formed by multiplying a reference anatomic image by images of the needle in a uniform background at various positions. The dictionary can also include spatial distortion maps that describe signal attenuation and spatial shifts that result from the presence of the device in various positions. These spatial distortion maps can be applied to the reference image to form dictionary entries. These images can be simulated based on a finite number of possible positions of the device and known or estimated susceptibility properties of the device. The simulated images may be further based on pulse sequence parameters of the imaging protocol. To estimate the device position, the acquired distorted image can be compared to the dictionary entries to determine the best match, which provides an initial estimate of the position of the device.

In other implementations, the position can be estimated based on a known previous location of the device. For example, the position estimate can be set as the previous position of the device. The estimate may also be set as a modified previous position. For example, the previous position can be modified by some measure of movement of the interventional device, such as a known or estimated motion taken by a robotic device or surgeon since the previous position was determined.

In some implementations, process block 104 also includes determining a model representation of the device position. The model representation of the device may be based on a known shape of the device. The model representation may be further based on known bending properties of the needle in some implementations.

The position may be described or modeled by a set of differentiable functions. Representing the position of the device using differentiable functions may be advantageous for the process of determining the accurate position of the device. For example, the use of differentiable functions may allow for gradient-based methods to be used in the optimization process that will be described later (e.g., in the context of process block 110). As one non-limiting example, the position may be described by a set of Gaussian functions. Thus, generating the model can include calibrating parameters of each Gaussian function. For example, the amplitude, standard deviation or width, and relative center of each Gaussian can be tuned to represent the device's original (e.g., unbent) shape. As another example, the position may be described by a set of bump functions or other differentiable functions, or a mixture of different types of differentiable functions. Moreover, flexible devices that can sustain deformations in use can be described by Gaussian or other functions that can shift in space with respect to one another. In some implementations, the known mechanical properties of the device may be used to constrain the device model. As a non-limiting example, the needle can be allowed to bend at known locations or be constrained by the amount the needle is able to bend.

In some implementations, the model used to represent the position can be heuristically determined. For example, the number of differentiable functions (e.g., Gaussian functions) can be tuned to balance computational complexity with accurate representation of the device. In other implementations, the model may be determined by selecting the sparsest representation that satisfies an accuracy or other constraint (e.g., <10% error). For example, the Gaussian model can be calibrated by reducing error between the Gaussian model and a point cloud representation of the needle while encouraging sparsity of the model. As another example, a Gaussian model can be used to describe a susceptibility map of the needle. For example, the Gaussian model can be fit to a point cloud representation of the needle shape that is convolved with a susceptibility Green's function, where each point of the needle can be treated as a delta function. As a non-limiting example, a straight needle may be represented by a mixture of 64 Gaussian functions. As another non-limiting example, a curved or flexible needle may be represented by thousands of Gaussian functions.

A magnetic susceptibility map can be calculated in process block 106. This susceptibility map can be based on the estimated, pre-characterized or known susceptibility properties of the device and the location estimate, as determined in process block 104 or a previous iteration (e.g., process block 110). The susceptibility properties of the device may be described as a susceptibility distribution in space based on the predetermined materials of the device (e.g., titanium, stainless steel, cobalt-based alloys, magnesium alloys, vitallium, iridium-platinum alloys, nickel alloys, polyurethane, brass, copper, zinc, and so forth). In some implementations, the magnetic susceptibility map can be modeled using a set of differentiable function, such as Gaussian functions, as similarly described for the model representation of the device shape. For example, a Gaussian model can be fit to describe the susceptibility of the device, as previously described. In some implementations, a model that describes the susceptibility map can be calibrated empirically based on additional calibration measurements. For example, the device can be imaged in a well-characterized phantom to determine the susceptibility map that describes the device.

A simulated image or a k-space representation of a simulated image can be simulated in process block 108 based on the susceptibility map generated in process block 106. The simulated image may be a real-valued (e.g., magnitude) or complex-valued (e.g., magnitude and phase) image. Generating the simulated MR image in process block 108 can also be based on the pulse sequence parameters that were used to acquire the distorted image. The simulated image can be simulated by modifying a reference image that represents the acquired image without distortion caused by the presence of the device. In some implementations, the reference image can include a ground truth image. For example, a ground truth image can be acquired from the subject prior to placing the device. In other implementations, the reference image can be estimated based on the distorted image. For example, the region of signal dropout and distortion can be masked by replacing the pixel values with an average pixel value in the surrounding region. For example, for a DBS lead placed in gray matter, the masked signal dropout region can be reassigned to have the signal values based on the average signal in the gray matter region. As another example, other in-painting algorithms, which may include artificial intelligence algorithms, can be used to produce an estimated reference image.

Generating the simulated MR image in process block 108 can include determining a simulated magnetic field distortion map. Such field distortion map can be generated based on the magnetic susceptibility map generated in process block 106. Generating the field distortion map based on the magnetic susceptibility map can be performed using a dipole field convolution method, which can calculate frequency or field distortions using the susceptibly map. In some implementations, determining the magnetic field distortion map includes convolving the spatially differentiable functions that describe the position of the interventional device with a Green's function that relates the susceptibility to magnetic field distortions. For example, the Gaussian susceptibility model can be finely sampled at a set of spatial locations and numerically convolved with the Green's function. In other implementations, analytical convolution of the susceptibility model with the susceptibility-to-field offset Green's functions.

In some implementations, the magnetic field distortion map can be further based on known field parameters (e.g., the nominal field strength or applied magnetic gradients) or a measured or estimated field inhomogeneity (e.g., determined using B₀ field mapping techniques).

Several different simulation methods and software packages can be used to produce a simulated image based on the object model and pulse sequence parameters. For example, in some implementations, a spatial distortion map can be generated based on the field distortion map and associated pulse sequence parameters. The spatial distortion map can be applied to the reference image by shifting the pixels or sub-sampled pixels, accordingly, to produce a simulated image. In some implementations, the simulated image can be produced by multiplying the reference image with a distortion image that is produced for a uniform background. In other implementations, the distortion can be removed from the acquired image by dividing out the simulated distortion and calculating a metric of signal homogeneity. The simulated image data can similarly be produced in k-space. For example, Bloch simulations can be used to calculate the k-space signal at each time point based on reference data or baseline data acquired without the presence of the device.

The spatial distortion map can describe a pixel-wise or sub-pixel-wise 3-dimensional shift for each position in space. In some implementations and for some pulse sequences, the spatial distortion map can be determined as

$${D}_{RO} ( x , y , z ) = \frac{Δ {B}_{0} ( x , y , z )}{{BW}_{RO}} ,$$

where D_{RO}(x, y, z) describes a spatially varying shift in the readout direction in pixels, ΔB₀ (x, y, z) describes the field distortion map or field inhomogeneity in Hz, and BW_{RO} describes the readout bandwidth in Hz/pixel. The shift can similarly be described and calculated for the slice directions (e.g., in 2D imaging) and the phase encoding direction (e.g., in echo planar imaging).

The signal amplitude of the simulated image may also be adjusted for signal loss caused by intravoxel phase dispersion. For example, the intravoxel phase dispersion can be estimated based on the spatially varying susceptibility map or the magnetic field map. In some implementations, the signal dropout can be included by simulating the image at a spatial resolution higher than that of the acquired image. Integrating over the voxel widths allows for appropriate signal cancelation in the final simulated image. In other implementations, the signal dropout can be included analytically by calculating the linear phase gradient experience by each voxel. The attenuated signal amplitude can be calculated based on the Fourier transform of the voxel basis function (e.g., a rectangular voxel in image space as a sinc function in k-space), evaluated at the k-space corresponding to the slope of that phase gradient.

In other implementations, the image can be simulated in process block 108 using the magnetic field distortion map and pulse sequence parameters. For example, the image can be simulated using a Bloch equation simulation. The Bloch equation simulation can be used to simulate a distorted image based on some estimate of the ground truth image (e.g., the reference image). In some implementations, the simulation can be used to modify the reference image according to the distortion caused by the interventional device that is arranged at the estimated position.

In some implementations, generating the simulated image can include accounting for altered excitation profiles (e.g., slice selection, excitation, refocusing, inversion, saturations). For example, a full spin echo pulse sequence can be simulated, including the excitation and inversion pulses, which affect an altered set of spins according to the off resonance caused by the device.

In some implementations, simulating the image can also include simulating the effects of undersampling, thereby producing an aliased image. In this way, the simulated image can be compared to the imaging data as acquired, prior to applying parallel imaging reconstruction.

Simulating the image in process block 108 may also include downsampling the simulated image in order to match the acquired resolution. For example, to more precisely simulate sub-pixel distortion or intravoxel signal loss, the susceptibility map or field map may be upsampled to a resolution higher than that of the distorted or reference images. As another example, the simulated image can be simulated as though it were acquired at a higher resolution. The simulated image can be simulated at this higher resolution and then downsampled to match the acquired resolution.

Once a simulated image has been generated in process block 108, the estimated device position can be updated in process block 110. If the estimated device position is described by differential functions (e.g., a set of Gaussian functions), process block 110 can include gradient-based optimization methods. The optimization may include repeating the process, as in process block 112, until a sufficient match between the simulated and acquired images is reached. In this way, each iteration can provide a search direction that reduces the difference between the simulated and acquired images. The simulated image can be updated accordingly, and the process repeated until a sufficient match is achieved or for a preset number or iterations.

In other implementations, process block 110 can include methods suitable for non-differentiable optimization problems, such as pattern search, finite differences, dictionary search, and so forth. Such optimization can include repeating the process, as in process block 112. For example, a new dictionary can be generated based on a previously estimated position to refine the dictionary search.

In general, updating the position of the device can be performed by comparing the simulated image generated in process block 108 with the acquired distorted image accessed in process block 102. Such comparison can be performed using magnitude or complex (e.g., magnitude and phase) imaging data.

In some implementations, the comparison can be performed on a subset of the imaging field of view (FOV) that focuses on the region surrounding the interventional device. In some implementations, the comparison can be performed on modified images. For example, each image (e.g., the simulated image and the acquired image) could be modified by taking a ratio or computing the complex difference between the image and a reference image (e.g., an image of the same anatomy prior to the placement of the device). In some implementations, the simulated image and acquired image can be compared in their corresponding k-space representations.

In some implementations, the optimization procedure includes generating an objective function that describes an error between the distorted MR image and the simulated MR image. If the device position is differentiably described, the objective function can advantageously be differentiable. Thus, the derivative of the objective function can be calculated to reduce the objective function.

In some implementations, the position estimation can be performed in process block 110 using a dictionary search or some combination of optimization methods and dictionary search. For example, a gradient-based optimization can be used to determine the linear displacement and rotation of the device, followed by a dictionary search to match the shape with a set of possible deformations. In this way, optimization techniques and dictionary searches can be used together to determine various aspects of the device position (e.g., linear displacement, rotation, and deformation).

In some implementations, the optimization process includes back-propagating gradients and updating the estimated position using a limited-memory Broyden-Fletcher-Goldfarb-Shanno (BFGS) algorithm. This algorithm approximates a Hessian matrix in order to accelerate convergence of the overall updates towards reducing the cost function. However, other optimization algorithms can also be used. The optimization algorithm can take derivatives as an input to update the needle parameters.

As a non-limiting example, the following method can be used to update the device position when described by a set of Gaussian functions. An objective function can be defined that can be minimized or reduced to determine needle parameters that describe the position (e.g., linear position, orientation, and deformation). The real- or complex-valued simulated image is denoted as Ĩ( [symbol] ; θ), where θ includes the parameters that describe the needle position. For example, 0 may include the center or mean of each Gaussian function, the covariance matrix of each Gaussian function, and additional group centers/means or a covariance matrix that represents affine motion of the device. I( [symbol] ) can be used to denote the real- or complex-valued distorted image that was acquired. The objective function can be described by the form:

$${\sum }_{i = 1}^{N} {\vert \tilde{I} ( {\vec{x}}_{i} ; θ ) - I ( {\vec{x}}_{i} ) \vert}^{2} + R ( θ )$$

Here, N describes the number of voxels used in the error calculation (e.g., the number of voxels in the FOV or in a region of interest surrounding the device), and R(θ) describes an optional regularization. The regularization can incorporate prior knowledge about the device and its position in order to guide the optimizer away from infeasible solutions or local minima. For example, R(θ) may penalize the amplitudes or vector norm of parameters or subsets of parameters to discourage large deviations from the initial needle position or a previous needle position. R(θ) could also be used to enable some flexing of the device by penalizing the differences between nearby Gaussians to encourage smooth deformations of the device. Such regularization discourages large deformations of the device.

As shown, the sum of squared differences between the simulated and observed images can be used, which is consistent with additive Gaussian noise processes in MRI. However, other error or goodness measures can be used, such as the sum of absolute or non-squared differences. As another example, the correlation between simulated and measured images could be maximized.

In some implementations, I( [symbol] ) and Ĩ( [symbol] ᵢ; θ) represent complex-valued acquired and simulated images, respectively. In this way, the optimization can consider both the phase and magnitude of the system, which may advantageously provide higher accuracy in the position update. However, in other implementations, I( [symbol] ) and Ĩ( [symbol] ᵢ; θ) represent real-valued images, which may advantageously reduce computational complexity.

Once the optimization process is complete, the position of the device can be output, as in process block 114. Outputting the position may include displaying the updated position of the device to a user on a user interface. For example, the needle position may be displayed overlaid on the acquired distorted image or on the reference or ground truth image. As another example, the needle position may be displayed on a pre-segmented image, simulated high resolution image, pre-treatment image, or a functional map, such as an ablation dose map. In some implementations, the needle position can be communicated to a robotic system that can be controlled accordingly to move the device to a desired location. The device position can be displayed using the device model (e.g., displaying the device according to a set of Gaussian functions). In other implementations, the device model can be related back to the mask or point cloud representation of the needle to display the needle at its estimated position. In some implementations, the image displayed can include the acquired image after distortion correction has been applied based on the determined position and the modeled susceptibility of the object.

In some implementations, process 100 can be expanded to include generating multiple images with different contrasts or pulse sequence parameters. In this way, accessing the distorted image in process block 102 can include accessing multiple images (e.g., different contrasts, pulse sequence timings, frequency encoding directions) that are acquired with the device arranged in the same position in each of the images. When multiple images are acquired, the optimization problem can be expanded to include information from each acquired image. For example, the objective function can be written as:

$${\sum }_{i = 1}^{N} {\vert {\tilde{I}}_{1} ( {i}_{} ; θ ) - {I}_{1} ( {i}_{} ) \vert}^{2} + {\vert {\tilde{I}}_{2} ( {i}_{} ; θ ) - {I}_{2} ( {i}_{} ) \vert}^{2} R ( θ )$$

where Ĩ₁ and Ĩ₂ represent two different images in which the device is equally arranged (e.g., sharing parameters θ) in each. In other implementations, the process 100 can be performed using a single distorted image, and the resulting position can be applied to additional images. For example, process 100 can be performed for a T₁-weighted image in order to determine the position of the device. Once a position of the device is determined, it can be displayed on a T₂-weighted image, or an image with other contrast (e.g., T₂-weighted, diffusion, and so forth) can be corrected via simulation based on the device position accordingly.

In some implementations, the optimization previously described can similarly be performed based on field map data. FIG. 1B describes a process 150 that is similar to process 100 outlined in FIG. 1A. However, the optimization process used to update the object position in space is based on simulated and measured field map data rather than (e.g., anatomical) images. In some implementations, process 150 may be advantageous as it does not rely on anatomical imaging data or use an estimate of the imaging volume in the absence of the object.

In process 152, a distorted field map is accessed in process block 152. Process block 152 can include directly accessing a distorted field map, which may be referred to as a B₀ map or magnetic field distortion map, or generating a field map using a suitable field mapping method with field mapping data (e.g., two images acquired with two different echo times). This data is acquired in the presence of the object, which produces large distortions in the nominal magnetic field. Thus, the field map can be referred to as a distorted field map or measured field map.

In process block 154, the distorted field map can be used to estimate the position of the object in space. As a non-limiting example, the object can be estimated by thresholding the field inhomogeneity with respect to the main magnetic field (B₀). For example, regions of large inhomogeneity (e.g., above a predefined threshold) can be assumed to be occupied by the object. Once the object position is estimated, a susceptibility map can be generated for the estimated object in process block 106, as previously described.

In process block 158 a field distortion map can be simulated based on the estimated object position. This simulated magnetic field distortion map can be generated as previously described with respect to process block 108, which can be used to simulate field distortions caused by the object. Further, a reference field map can be measured or assumed. In some implementations, for example, it can be assumed that in the absence of the object, the background field is uniform (e.g., ΔB₀=0) since the field inhomogeneity caused by the object is typically much greater than other field inhomogeneities (e.g., caused by the patient). In other implementations a background field map can be assumed to be well-approximated by a low-order (e.g., polynomial) or spatially smooth function that can be fit. For example, such assumption can be incorporated into the regularization of process block 160 or otherwise incorporated into the optimization problem. In still other implementations, a reference field map can be acquired or measured before the object is introduced, providing a ground truth reference field map.

In process block 160, the object position can be updated by comparing the simulated field map with the measured distorted field map. Process block 160 may include methods similar to those described in the context of process block 110. However, in process block 160, the optimization can be based on a comparison of the simulated field map with the measured field map, rather than images that include the anatomy or otherwise reflect the underlying signal. As a non-limiting example, the objective function can be defined according to:

$${\sum }_{i = 1}^{N} {\vert ( {i}_{} ; θ ) - Δ {B}_{0} ( {i}_{} ) \vert}^{2} + R ( θ )$$

where [symbol] represents the field map simulated for an object at a position described by θ and ΔB₀ represents the measured field map. As previously described, the optimization can be repeated to iteratively update the object position, as in process block 112, until the objective function is sufficiently reduced or minimized. Once a stopping criterion is satisfied (e.g., the error is below a given threshold, or a predefined number of iterations has been performed) the position can be saved or output as described in block 114. In some implementation, the object's position can be displayed with respect to a magnitude image produced from the field mapping data accessed in process block 152.

### EXAMPLES

### Example 1

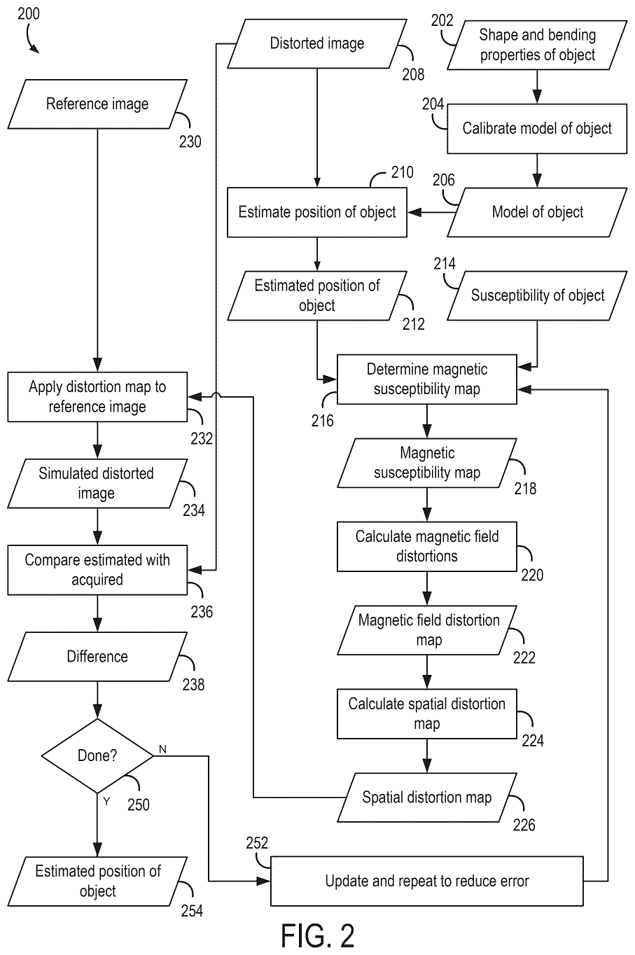

FIG. 2 provides a flowchart setting forth the steps of an example process 200 that can be used to determine the position of an object arranged in a subject during acquisition of an MR image. Thus, FIG. 2 provides additional details or sub-steps that can be used as a non-limiting example to perform the process 100 outlined in FIG. 1A.

Known properties 202 (e.g., shape and bending properties) of the object (e.g., needle or other interventional device) are used to calibrate a model representation 206 of the object in process block 204. In some implementations, the model representation 206 may include a set of differentiable functions. For example, the set of differentiable functions can include Gaussian functions, and calibrating the model can include setting the parameters of each Gaussian function. For example, calibrating the model can include setting the amplitude, relative center, and standard deviation of each Gaussian function.

In process block 210, this model 206 is used with an acquired distorted image 208 to estimate the position 212 of the object in the distorted image 208. Estimating the position may include tuning parameters that describe the position 212 of set of differentiable functions. For example, the parameters that describe the position may include a mean shift that describes the linear position of the model representation 206. The parameters may also include a rotation or rotation matrix that describes the orientation of the model representation 206. In some implementations, the parameters can further include linear shifts of each function in the set of functions to include deformation in the model representation 206.

In process block 216, a magnetic susceptibility map 218 can be generated for the object located at the estimated position 212 using the known susceptibility properties of the object 214. The magnetic susceptibility map 218 can be used in process block 220 to calculate a map of magnetic field distortions 222 caused by the susceptibility properties of the object 214.

The magnetic field distortions 222 can be used to generate a simulated image based on a reference image 230. In some implementations, generating the simulated image 234 includes determining a spatial distortion map 226, as in process block 224, that is based on the field distortion map 222 and the pulse sequence parameters used to acquire the distorted image 208. The spatial distortion map 226 may represent a spatial shift that can be applied to each pixel of a reference image 230 in process block 232 to produce a simulated distorted image 234. This simulated image 234 can also be modified based on an estimated signal loss caused by intravoxel dephasing. In other implementations, the simulated image 234 can be directly estimated (e.g., using Bloch equation simulation) using the magnetic field distortion map 222 and some reference image 230 that represents an undistorted image.

The simulated image 234 and acquired distorted image 208 can be compared in process block 236 in order to update the position of the object. As a non-limiting example, process block 236 can include evaluating an objective or loss function that is based on the difference 238 between the simulated image 234 and the acquired image 208. In other implementations, other goodness of fit measures (e.g., correlation, squared differences) can be used. The objective function can be assessed in process block 250 to determine whether the difference 238 between the simulated image 234 and the acquired image 208 is sufficiently small. If not, the position can be updated and the process repeated, as in process block 252. In this way, the process can be iteratively repeated until the difference 238 between these images or other loss function is sufficiently reduced or minimized. Once complete, the estimated position 254 can be used for clinical care by displaying the needle position to a clinician or using the estimated position 254 to guide intervention. The estimated position of the object 254 can also be used to simulate undistorted images in which the artifacts caused by the implanted device are corrected.

### Example 2

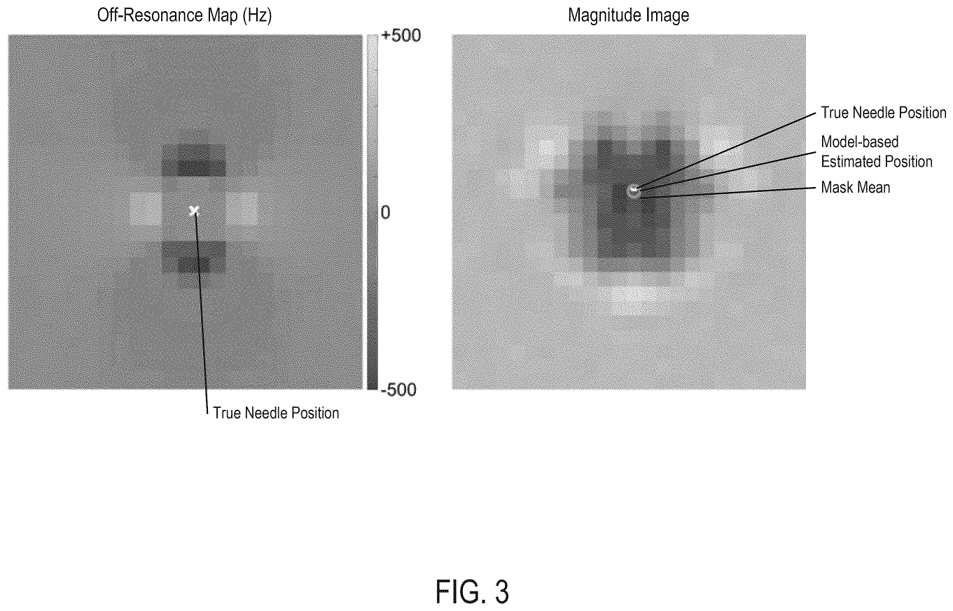

FIG. 3 shows a simulated 2D needle localization with a gradient-recalled echo sequence. The 4 mm-diameter nitinol needle is oriented perpendicular to the B₀ field and the frequency encoded dimension, which are both in the vertical dimension. The readout bandwidth is 62 kHz with a 256 matrix size across a 20 cm FOV (0.78 mm² voxel size). The needle produces several hundred-Hz frequency offsets which lead to asymmetric blurring in the frequency encoded dimension as well as dispersion-induced signal loss. Thresholding the magnitude image and taking the mean coordinates of the resulting mask leads to approximately 0.5 mm error in the position of the needle; much larger errors are typical in 3D measurements. The model-based estimated position was provided by the disclosed method. The described systems and methods correctly determine the needle position by correctly modeling the image distortion produced by the needle and determining the position via the process of aligning the distorted and acquired images.

### Example 3

The following non-limiting example validates the described methods using simulations compared to a PCA-based approach. In this example, the described methods achieved up to 50% lower position and angular errors than the PCA approach.

### Motivation

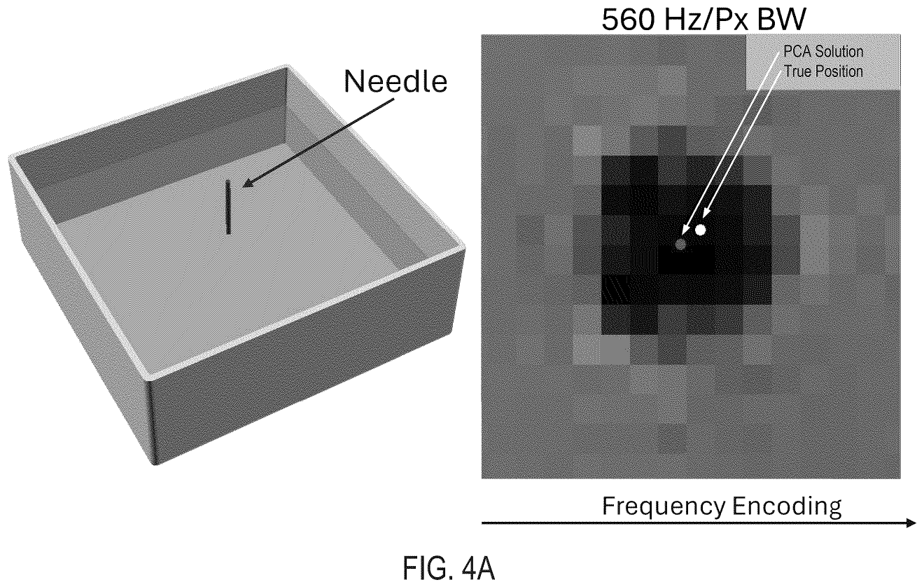

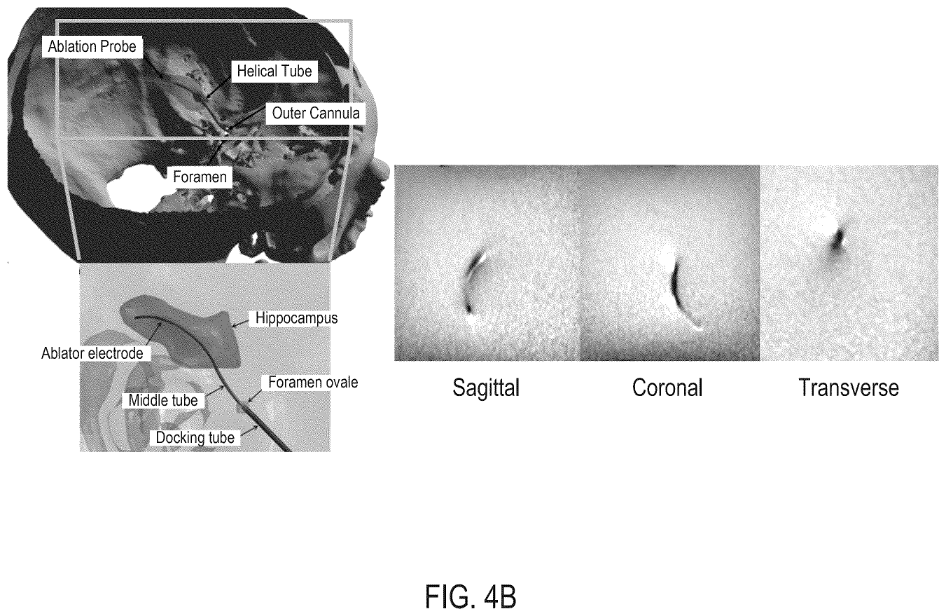

FIG. 4A provides an experimental demonstration of a 1.1 mm OD curved nitinol needle image distortion and image-based position error using a PCA approach. As illustrated in FIG. 4A, the distortions and subsequently estimated needle positions depend on the orientation of the needle with respect to B₀, as well as sequence parameters such as pixel bandwidth. Further, as illustrated in FIG. 4B, curved needles are the best choice for procedures such as transforamenal hippocampal ablation to treat epilepsy, but current image-based needle localization methods only apply to straight inflexible needles and do not apply to curved needles, which present more complicated image distortions. Moreover, current methods cannot account for possible needle flexing, such as in needle-based transforaminal hippocampal ablation surgery. The described systems and methods can be applied in the context of curved or flexed needles.

### Methods

### Needle Modeling and Distorted Image Calculation

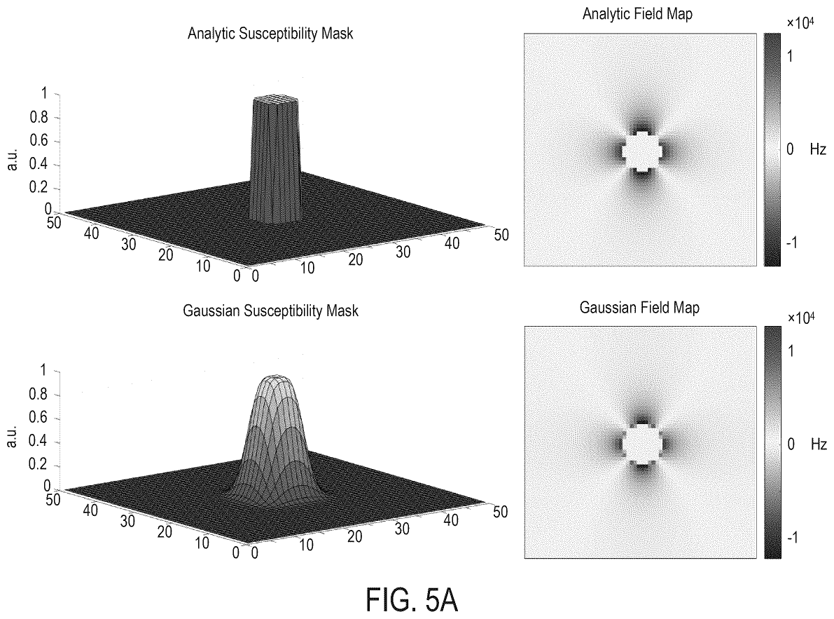

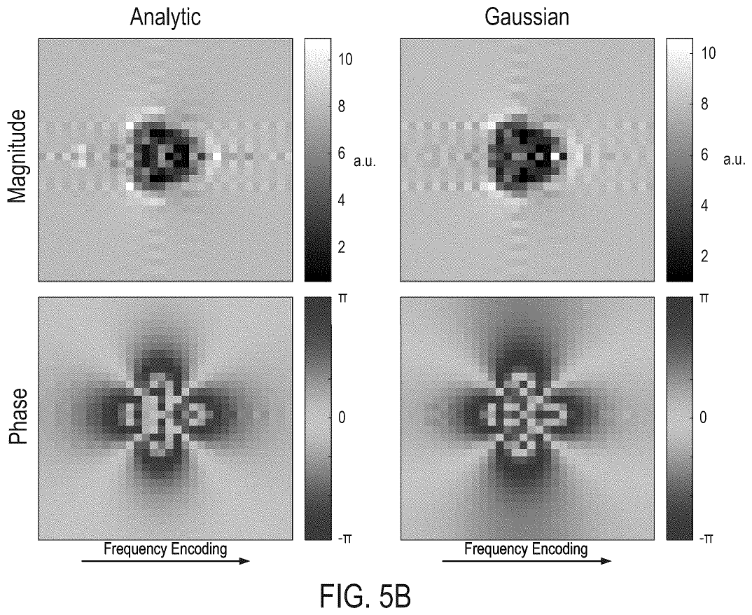

Inspired by the Gaussian splatting technique in computer graphics, the needle's susceptibility was represented as a spatial mixture of Gaussian functions that are parameterized by their amplitudes, positions, and widths or standard deviations, all of which can be continuously differentiated. FIG. 5A illustrates this model compared to a mask-based analytic cylindrical needle representation. FIG. 5A shows a 2D slice through non-differentiable mask-based and differentiable Gaussian needle susceptibility models, and the corresponding calculated field maps.

The field map was calculated by convolving the Gaussians by the susceptibility Green's function. Distorted images are then calculated using the field map and the sequence's specific echo time and pixel bandwidth. The final images are downsampled to the acquired resolution, to capture intravoxel effects. FIG. 5B shows that the distorted image magnitude and phase from analytic and differentiable Gaussian models match well.

### Gradient-Based Needle Position and Pose Recovery

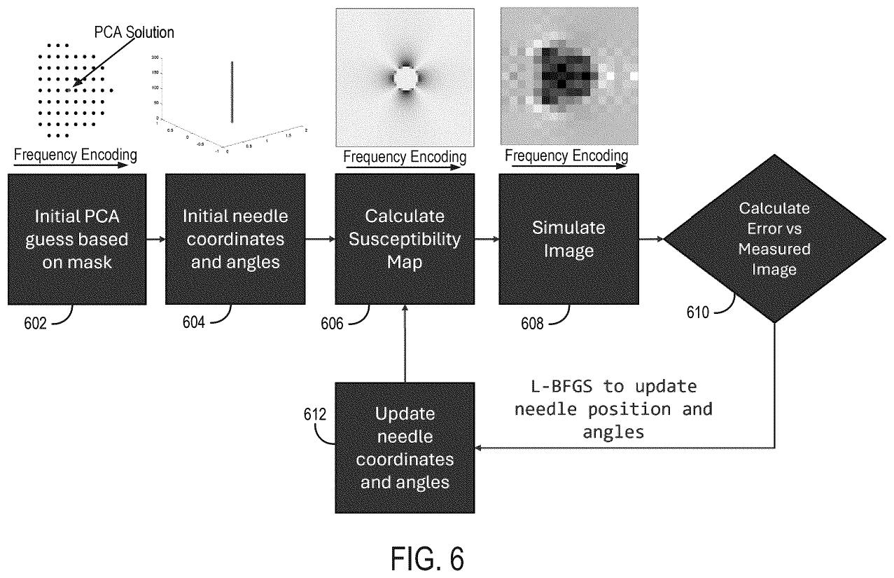

FIG. 6 illustrates an optimization procedure to recover the needle position (e.g., displacement, orientation, and flexing) from an acquired image. The needle location is first estimated in process block 602 by using principal component analysis (PCA) to find the center of the 3D image distortion caused by the needle and its approximate angle of rotation 604. This initial guess 604 is used to initialize the differentiable (e.g., Gaussian) model to generate a susceptibility map, as in process block 606, which is used to calculate a field map and a simulated distorted image 608. The simulated image 608 is compared to the measured image via a loss function in process block 610. Gradients are back-propagated and the Torch L-BFGS algorithm calculates updates to the needle position and pose parameters in process block 612, to achieve a match between the images. In this way, the needle coordinates and orientation are updated using an iterative solver to minimize the loss, leveraging differentiability of the entire forward model.

FIG. 6 also provides non-limiting example outputs of the steps set forth. Element 622 represents an initially estimated position of a needle generated using a PCA. The needle can be represented in space at this position, as shown in element 624. A susceptibility map associated with this needle, which is modeled in space using a collection of Gaussian functions, is shown in element 626. When this susceptibility map is applied to simulate the image, the resulting distorted image is shown in element 628.

### Simulations

A straight needle was simulated, and subsequently shifted −0.25 mm in the x direction and 0.75 mm in z and rotated 10°. The susceptibility map and distorted image of the shifted and rotated needle were then calculated. This served as the target image for matching. The void in the simulated image was used to calculate a PCA estimate of the shifts and rotations of the needle, which were used to initialize the optimizer described in FIG. xx. 2 epochs of 100 iterations with a learning rate of 0.1 were used in the optimization in this non-limiting example.

### Results

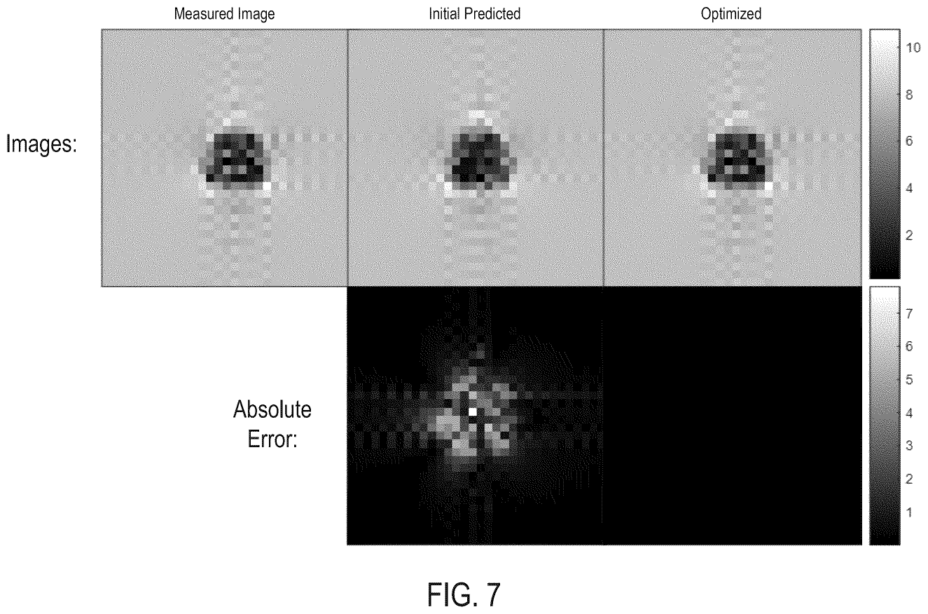

FIG. 7 shows simulation data for the needle localization on the cross section of a shifted and rotated needle. The top row shows a slice through the needle of the target image (left), the initial shift and rotation found by PCA (middle), and the final results from the optimizer (right). The bottom row shows the absolute error between the PCA solution and the target, and the error between the final optimized output and the target.

The target shift and rotation were (−0.25, 0.75) mm and 10°. The initial PCA estimates were (−0.382, 0.4699) mm shift and a 13.47° rotation. The optimizer output found an in-plane shift of (−0.2563, 0.7443) mm, and 10.26° rotation in one axis.

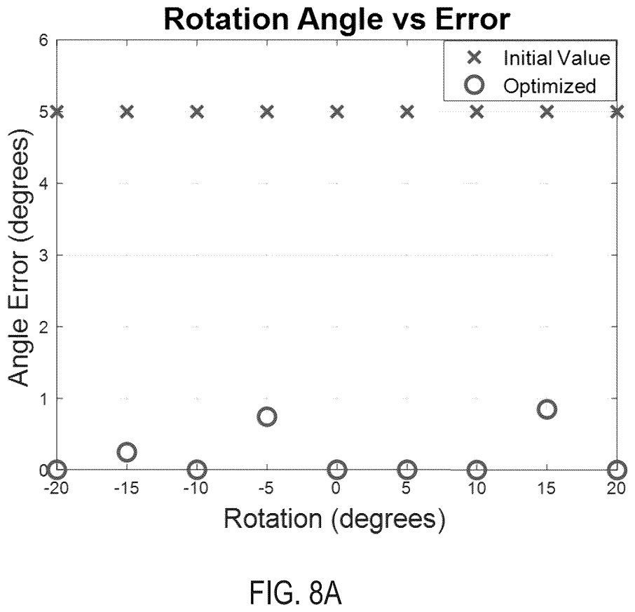

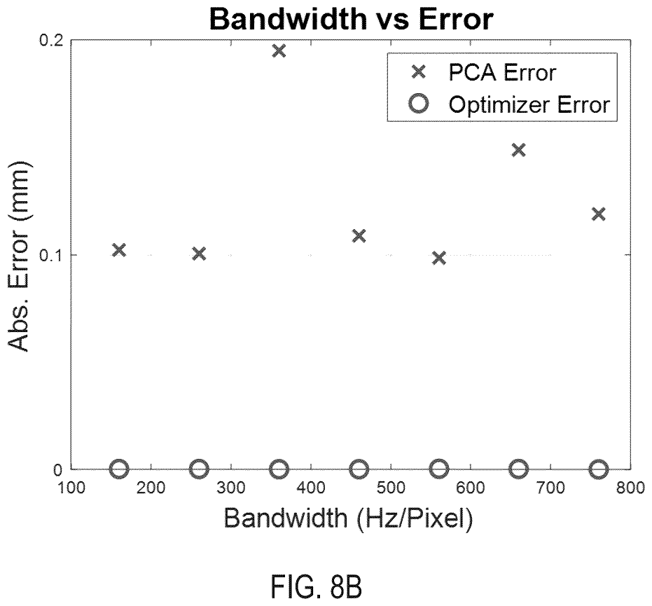

FIG. 8A shows the result of the optimizer identifying rotations in one axis ranging from −20 to +20 degrees. The optimizer was initialized with an angle 5 degrees away from the target value in each case. Across angles, the results were under 1 degree of error. FIG. 8B shows the results of the optimized output solving for a −0.4 mm shift in the frequency encoding dimension, across bandwidths ranging from 160 Hz/Pixel to 760 Hz/Pixel. The optimizer was initialized with PCA results, which are also provided. The optimizer was robust to changes in bandwidth and had less than 0.1 mm error in each case. In the results shown in FIGS. 8A and 8B, 1 epoch of 100 iterations with a learning rate of 0.1 was used.

### Discussion and Conclusion

The described example provides a differentiable model for an interventional device and its effect on MR images, which enables the recovery of a needle's position and pose based on image distortions. In this example, results were simulated using a straight needle. However, the described methods can also be used with straight and curved needles in various orientations and with various needle deformations. Modeling the needle with a spatial mixture of Gaussians provides particular flexibility in the needle form and possible deformations that occur during use.

### Example Systems

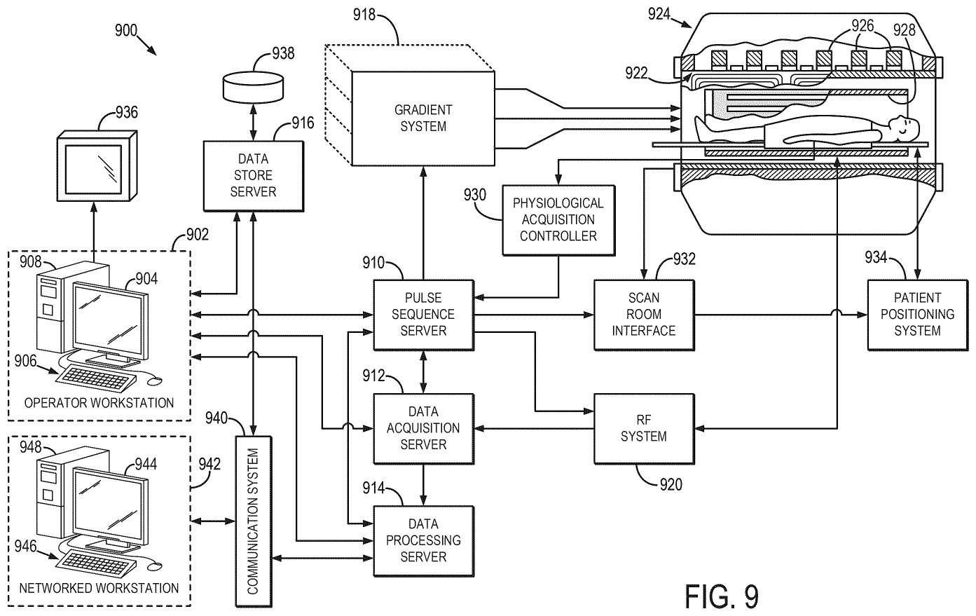

Referring particularly now to FIG. 9, an example of an MRI system 900 that can implement the methods described herein is illustrated. The MRI system 900 includes an operator workstation 902 that may include a display 904, one or more input devices 906 (e.g., a keyboard, a mouse), and a processor 908. The processor 908 may include a commercially available programmable machine running a commercially available operating system. The operator workstation 902 provides an operator interface that facilitates entering scan parameters into the MRI system 900. The operator workstation 902 may be coupled to different servers, including, for example, a pulse sequence server 910, a data acquisition server 912, a data processing server 914, and a data store server 916. The operator workstation 902 and the servers 910, 912, 914, and 916 may be connected via a communication system 940, which may include wired or wireless network connections.

The MRI system 900 also includes a magnet assembly 924 that includes a polarizing magnet 926, which may be a low-field magnet. The MRI system 900 may optionally include a whole-body RF coil 928 and a gradient system 918 that controls a gradient coil assembly 922.

The pulse sequence server 910 functions in response to instructions provided by the operator workstation 902 to operate a gradient system 918 and a radiofrequency (“RF”) system 920. Gradient waveforms for performing a prescribed scan are produced and applied to the gradient system 918, which then excited gradient coils in an assembly 922 to produce the magnetic field gradients (e.g., Gₓ, G_{y}, and G_{z}) that can be used for spatially encoding magnetic resonance signals. The gradient coil assembly 922 forms part of a magnet assembly 924 that includes a polarizing magnet 926 and a whole-body RF coil 928.

RF waveforms are applied by the RF system 920 to the RF coil 928, or a separate local coil to perform the prescribed magnetic resonance pulse sequence. Responsive magnetic resonance signals detected by the RF coil 928, or a separate local coil, are received by the RF system 920. The responsive magnetic resonance signals may be amplified, demodulated, filtered, and digitized under direction of commands produced by the pulse sequence server 910. The RF system 920 includes an RF transmitter for producing a wide variety of RF pulses used in MRI pulse sequences. The RF transmitter is responsive to the prescribed scan and direction from the pulse sequence server 910 to produce RF pulses of the desired frequency, phase, and pulse amplitude waveform. The generated RF pulses may be applied to the whole-body RF coil 928 or to one or more local coils or coil arrays.

The RF system 920 also includes one or more RF receiver channels. An RF receiver channel includes an RF preamplifier that amplifies the magnetic resonance signal received by the coil 928 to which it is connected, and a detector that detects and digitizes the I and Q quadrature components of the received magnetic resonance signal. The magnitude of the received magnetic resonance signal may, therefore, be determined at a sampled point by the square root of the sum of the squares of the I and Q components:

$$M = \sqrt{( {I}^{2} + {Q}^{2} )}$$

and the phase of the received magnetic resonance signal may also be determined according to the following relationship:

$$ϕ = {tan}^{- 1} ( \frac{Q}{I} )$$

The pulse sequence server 910 may receive patient data from a physiological acquisition controller 930. By way of example, the physiological acquisition controller 930 may receive signals from a number of different sensors connected to the patient, including electrocardiograph (“ECG”) from electrodes, or respiratory signals from a respiratory bellows or other respiratory monitoring devices. These signals may be used by the pulse sequence server 910 to synchronize, or “gate,” the performance of the scan with the subject's heartbeat or respiration.

The pulse sequence server 910 may also connect to a scan room interface circuit 932 that receives signals from various sensors associated with the condition of the patient and the magnet system. Through the scan room interface circuit 932, a patient positioning system 934 can receive commands to move the patient to desired positions during the scan.

The digitized magnetic resonance signal samples produced by the RF system 920 are received by the data acquisition server 912. The data acquisition server 912 operates in response to instructions downloaded from the operator workstation 902 to receive the real-time magnetic resonance data and provide buffer storage, so that data are not lost by data overrun. In some scans, the data acquisition server 912 passes the acquired magnetic resonance data to the data processor server 914. In scans that require information derived from acquired magnetic resonance data to control the further performance of the scan, the data acquisition server 912 may be programmed to produce such information and convey it to the pulse sequence server 910. For example, during pre-scans, magnetic resonance data may be acquired and used to calibrate the pulse sequence performed by the pulse sequence server 910. As another example, navigator signals may be acquired and used to adjust the operating parameters of the RF system 920 or the gradient system 918, or to control the view order in which k-space is sampled. In still another example, the data acquisition server 912 may also process magnetic resonance signals used to detect the arrival of a contrast agent in a magnetic resonance angiography (“MRA”) scan. For example, the data acquisition server 912 may acquire magnetic resonance data and processes it in real-time to produce information that is used to control the scan.

The data processing server 914 receives magnetic resonance data from the data acquisition server 912 and processes the magnetic resonance data in accordance with instructions provided by the operator workstation 902. Such processing may include, for example, reconstructing two-dimensional or three-dimensional images by performing a Fourier transformation of raw k-space data, performing other image reconstruction algorithms (e.g., iterative or backprojection reconstruction algorithms), applying filters to raw k-space data or to reconstructed images, generating functional magnetic resonance images, or calculating motion or flow images.

Images reconstructed by the data processing server 914 are conveyed back to the operator workstation 902 for storage. Real-time images may be stored in a data base memory cache, from which they may be output to operator display 902 or a display 936. Batch mode images or selected real time images may be stored in a host database on disc storage 938. When such images have been reconstructed and transferred to storage, the data processing server 914 may notify the data store server 916 on the operator workstation 902. The operator workstation 902 may be used by an operator to archive the images, produce films, or send the images via a network to other facilities.

The MRI system 900 may also include one or more networked workstations 942. For example, a networked workstation 942 may include a display 944, one or more input devices 946 (e.g., a keyboard, a mouse), and a processor 948. The networked workstation 942 may be located within the same facility as the operator workstation 902, or in a different facility, such as a different healthcare institution or clinic.

The networked workstation 942 may gain remote access to the data processing server 914 or data store server 916 via the communication system 940. Accordingly, multiple networked workstations 942 may have access to the data processing server 914 and the data store server 916. In this manner, magnetic resonance data, reconstructed images, or other data may be exchanged between the data processing server 914 or the data store server 916 and the networked workstations 942, such that the data or images may be remotely processed by a networked workstation 942.

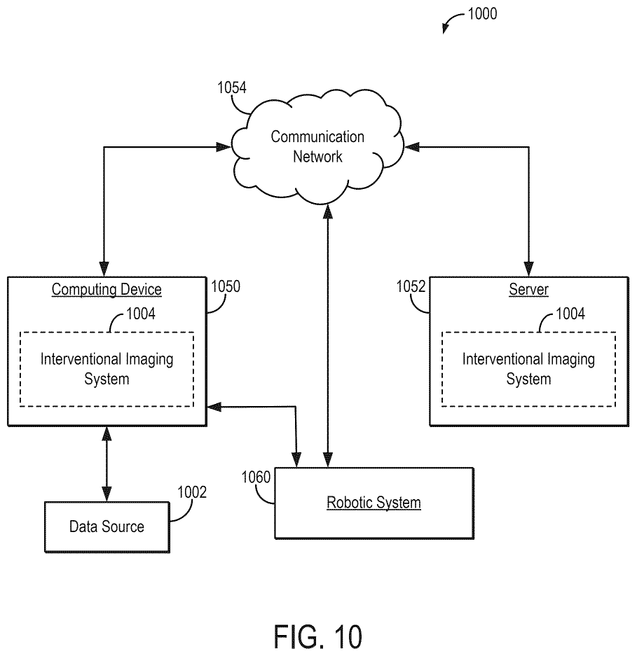

Referring now to FIG. 10, an example of a system 1000 is shown, which may be used in accordance with some aspects of the systems and methods described in the present disclosure. As shown in FIG. 10, a computing device 1050 can receive one or more types of data (e.g., signal evolution data, k-space data, receiver coil sensitivity data, interventional device property data, model representation of an interventional device) from data source 1002. In some configurations, computing device 1050 can execute at least a portion of an interventional imaging system 1004 to reconstruct images from magnetic resonance data (e.g., k-space data) acquired in the presence of an interventional device and estimate the position of the interventional device as previously described. In some configurations, the interventional imaging system 1004 can implement an automated pipeline to provide MRI images in which the distortion caused by the device is corrected, images on which the device position is displayed, a description of the position of the device, segmented or annotate images, etc.

Additionally or alternatively, in some configurations, the computing device 1050 can communicate information about data received from the data source 1002 to a server 1052 over a communication network 1054, which can execute at least a portion of the interventional imaging system 1004. In such configurations, the server 1052 can return information to the computing device 1050 (and/or any other suitable computing device) indicative of an output of the interventional imaging system 1004.

In some configurations, computing device 1050 and/or server 1052 can be any suitable computing device or combination of devices, such as a desktop computer, a laptop computer, a smartphone, a tablet computer, a wearable computer, a server computer, a virtual machine being executed by a physical computing device, and so on. The computing device 1050 and/or server 1052 can also reconstruct images from the data.

In some configurations, data source 1002 can be any suitable source of data (e.g., measurement data, images reconstructed from measurement data, processed image data, properties of the interventional device, user inputs, a model representation of an interventional device), such as an MRI system, another computing device (e.g., a server storing measurement data, images reconstructed from measurement data, processed image data), and so on. In some configurations, data source 1002 can be local to computing device 1050. For example, data source 1002 can be incorporated with computing device 1050 (e.g., computing device 1050 can be configured as part of a device for measuring, recording, estimating, acquiring, or otherwise collecting or storing data). As another example, data source 1002 can be connected to computing device 1050 by a cable, a direct wireless link, and so on. Additionally or alternatively, in some configurations, data source 1002 can be located locally and/or remotely from computing device 1050, and can communicate data to computing device 1050 (and/or server 1052) via a communication network (e.g., communication network 1054).

In some configurations, communication network 1054 can be any suitable communication network or combination of communication networks. For example, communication network 1054 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), other types of wireless network, a wired network, and so on. In some configurations, communication network 1054 can be a local area network, a wide area network, a public network (e.g., the Internet), a private or semi-private network (e.g., a corporate or university intranet), any other suitable type of network, or any suitable combination of networks. Communications links shown in FIG. 10 can each be any suitable communications link or combination of communications links, such as wired links, fiber optic links, Wi-Fi links, Bluetooth links, cellular links, and so on.

In some implementations, the system 1000 can include a robotic system 1060 that is in communication with the computing device 1050, either directly or via the communication network 1054. The robotic system can be used to robotically control an interventional device according to the images acquired, the estimated position of the interventional device, or a user input.

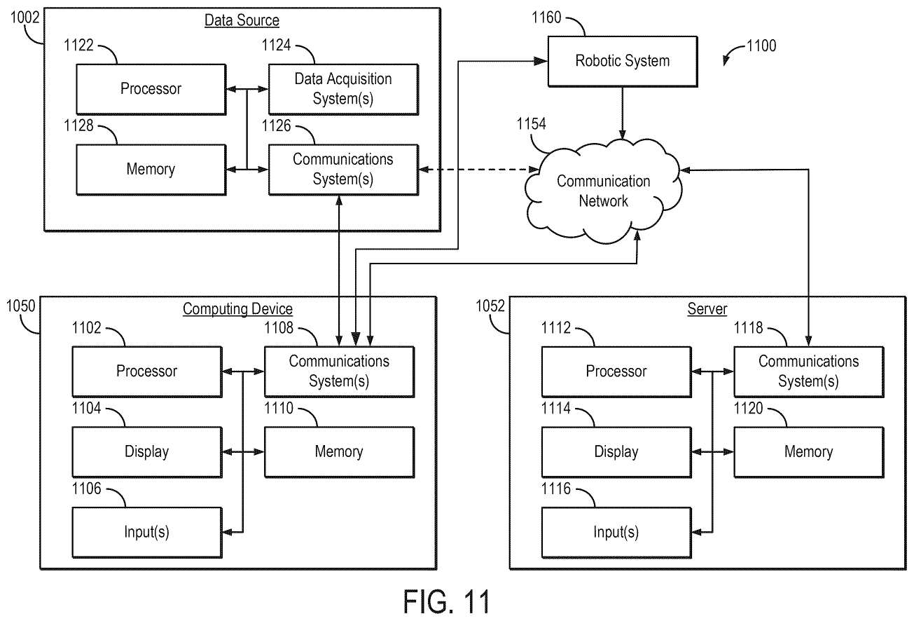

Referring now to FIG. 11, an example of hardware 1100 that can be used to implement data source 1002, computing device 1050, and server 1052 in accordance with some configurations of the systems and methods described in the present disclosure is shown.

As shown in FIG. 11, in some configurations, computing device 1050 can include a processor 1102, a display 1104, one or more inputs 1106, one or more communication systems 1108, and/or memory 1110. In some configurations, processor 1102 can be any suitable hardware processor or combination of processors, such as a central processing unit (“CPU”), a graphics processing unit (“GPU”), and so on. In some configurations, display 1104 can include any suitable display devices, such as a liquid crystal display (“LCD”) screen, a light-emitting diode (“LED”) display, an organic LED (“OLED”) display, an electrophoretic display (e.g., an “e-ink” display), a computer monitor, a touchscreen, a television, and so on. In some configurations, inputs 1106 can include any suitable input devices and/or sensors that can be used to receive user input, such as a keyboard, a mouse, a touchscreen, a microphone, and so on.

In some configurations, communications systems 1108 can include any suitable hardware, firmware, and/or software for communicating information over communication network 1054 and/or any other suitable communication networks. For example, communications systems 1108 can include one or more transceivers, one or more communication chips and/or chip sets, and so on. In a more particular example, communications systems 1108 can include hardware, firmware, and/or software that can be used to establish a Wi-Fi connection, a Bluetooth connection, a cellular connection, an Ethernet connection, and so on.

In some configurations, memory 1110 can include any suitable storage device or devices that can be used to store instructions, values, data, or the like, that can be used, for example, by processor 1102 to present content using display 1104, to communicate with server 1052 via communications system(s) 1108, and so on. Memory 1110 can include any suitable volatile memory, non-volatile memory, storage, or any suitable combination thereof. For example, memory 1110 can include random-access memory (“RAM”), read-only memory (“ROM”), electrically programmable ROM (“EPROM”), electrically erasable ROM (“EEPROM”), other forms of volatile memory, other forms of non-volatile memory, one or more forms of semi-volatile memory, one or more flash drives, one or more hard disks, one or more solid state drives, one or more optical drives, and so on. In some configurations, memory 1110 can have encoded thereon, or otherwise stored therein, a computer program for controlling operation of computing device 1050. In such configurations, processor 1102 can execute at least a portion of the computer program to present content (e.g., images, user interfaces, graphics, tables), receive content from server 1052, transmit information to server 1052, and so on. For example, the processor 1102 and the memory 1110 can be configured to perform the methods described herein.

In some configurations, server 1052 can include a processor 1112, a display 1114, one or more inputs 1116, one or more communications systems 1118, and/or memory 1120. In some configurations, processor 1112 can be any suitable hardware processor or combination of processors, such as a CPU, a GPU, and so on. In some configurations, display 1114 can include any suitable display devices, such as an LCD screen, LED display, OLED display, electrophoretic display, a computer monitor, a touchscreen, a television, and so on. In some configurations, inputs 1116 can include any suitable input devices and/or sensors that can be used to receive user input, such as a keyboard, a mouse, a touchscreen, a microphone, and so on.

In some configurations, communications systems 1118 can include any suitable hardware, firmware, and/or software for communicating information over communication network 1054 and/or any other suitable communication networks. For example, communications systems 1118 can include one or more transceivers, one or more communication chips and/or chip sets, and so on. In a more particular example, communications systems 1118 can include hardware, firmware, and/or software that can be used to establish a Wi-Fi connection, a Bluetooth connection, a cellular connection, an Ethernet connection, and so on.

In some configurations, memory 1120 can include any suitable storage device or devices that can be used to store instructions, values, data, or the like, that can be used, for example, by processor 1112 to present content using display 1114, to communicate with one or more computing devices 1050, and so on. Memory 1120 can include any suitable volatile memory, non-volatile memory, storage, or any suitable combination thereof. For example, memory 1120 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some configurations, memory 1120 can have encoded thereon a server program for controlling operation of server 1052. In such configurations, processor 1112 can execute at least a portion of the server program to transmit information and/or content (e.g., data, images, a user interface) to one or more computing devices 1050, receive information and/or content from one or more computing devices 1050, receive instructions from one or more devices (e.g., a personal computer, a laptop computer, a tablet computer, a smartphone), and so on.

In some configurations, the server 1052 is configured to perform the methods described in the present disclosure. For example, the processor 1112 and memory 1120 can be configured to perform the methods described herein.

In some configurations, data source 1002 can include a processor 1122, one or more data acquisition systems 1124, one or more communications systems 1126, and/or memory 1128. In some configurations, processor 1122 can be any suitable hardware processor or combination of processors, such as a CPU, a GPU, and so on. In some configurations, the one or more data acquisition systems 1124 are generally configured to acquire data, images, or both, and can include an MRI system. Additionally or alternatively, in some configurations, the one or more data acquisition systems 1124 can include any suitable hardware, firmware, and/or software for coupling to and/or controlling operations of an MRI system. In some configurations, one or more portions of the data acquisition system(s) 1124 can be removable and/or replaceable.

Note that, although not shown, data source 1002 can include any suitable inputs and/or outputs. For example, data source 1002 can include input devices and/or sensors that can be used to receive user input, such as a keyboard, a mouse, a touchscreen, a microphone, a trackpad, a trackball, and so on. As another example, data source 1002 can include any suitable display devices, such as an LCD screen, an LED display, an OLED display, an electrophoretic display, a computer monitor, a touchscreen, a television, etc., one or more speakers, and so on.

In some configurations, communications systems 1126 can include any suitable hardware, firmware, and/or software for communicating information to computing device 1050 (and, in some configurations, over communication network 1054 and/or any other suitable communication networks). For example, communications systems 1126 can include one or more transceivers, one or more communication chips and/or chip sets, and so on. In a more particular example, communications systems 1126 can include hardware, firmware, and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some configurations, memory 1128 can include any suitable storage device or devices that can be used to store instructions, values, data, or the like, that can be used, for example, by processor 1122 to control the one or more data acquisition systems 1124, and/or receive data from the one or more data acquisition systems 1124; to generate images from data; present content (e.g., data, images, a user interface) using a display; communicate with one or more computing devices 1050; and so on. Memory 1128 can include any suitable volatile memory, non-volatile memory, storage, or any suitable combination thereof. For example, memory 1128 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some configurations, memory 1128 can have encoded thereon, or otherwise stored therein, a program for controlling operation of medical image data source 1002. In such configurations, processor 1122 can execute at least a portion of the program to generate images, transmit information and/or content (e.g., data, images, a user interface) to one or more computing devices 1050, receive information and/or content from one or more computing devices 1050, receive instructions from one or more devices (e.g., a personal computer, a laptop computer, a tablet computer, a smartphone, etc.), and so on.

In some configurations, any suitable computer-readable media can be used for storing instructions for performing the functions and/or processes described herein. For example, in some configurations, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “controller,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

As used herein, the phrase “at least one of A, B, and C” means at least one of A, at least one of B, and/or at least one of C, or any one of A, B, or C or combination of A, B, or C. A, B, and C are elements of a list, and A, B, and C may be anything contained in the Specification.

The present disclosure has described one or more preferred embodiments, and it should be appreciated that many equivalents, alternatives, variations, and modifications, aside from those expressly stated, are possible and within the scope of the invention.

1. A method for generating a magnetic resonance (MR) image in the presence of an interventional device that induces distortions, the method comprising using a processor to perform steps comprising:
accessing a distorted MR image of a subject with the interventional device arranged in the subject;
determining, using the distorted MR image, parameters of a set of spatially differentiable functions describing an estimated position of the interventional device in the subject;
determining a magnetic susceptibility map based on susceptibility properties of the interventional device and the estimated position of the interventional device;
generating a simulated MR image by modifying a reference image of the subject based on the magnetic susceptibility map; and
updating the estimated position using the simulated MR image to generate an updated position of the interventional device in the subject.

2. The method of claim 1, wherein the interventional device is at least one of a needle, a biopsy clip, a deep brain stimulation lead, an electrode lead, a fiducial marker, a pacemaker, or an implant that has a susceptibility different than a susceptibility of biological tissue.

3. The method of claim 1, wherein the set of spatially differentiable functions comprises Gaussian functions.

4. The method of claim 1, wherein the position includes at least one of a linear position or an orientation.

5. The method of claim 4, wherein the position further includes a description of a deformation of the interventional device.

6. The method of claim 1, wherein the processor is further configured to perform steps including at least one of:
determining the set of spatially differentiable functions sufficient to represent the interventional device; displaying the updated position to a user; or
controlling robotic movement of the interventional device based on the updated position of the interventional device.

7. The method of claim 1, wherein the step of determining the parameters of the spatially differentiable functions comprises at least one of:
masking the distorted MR image according to a signal dropout caused by the interventional device and calculating a center of mass of the mask to determine the estimated position;
masking the distorted MR image according to a signal dropout caused by the interventional device and performing a principal component analysis on the mask to determine the estimated position;
forming a dictionary of a set of images in which the reference image is modified according to a finite number of possible positions of the interventional device and the susceptibility properties of the interventional device, and comparing the distorted MR image with each of the images in the dictionary to determine a best match to determine the estimated position;
accessing a known previous position of the interventional device to determine the estimated position; or
accessing a known previous position of the interventional device and a measure of movement of the interventional device, and modifying the known previous position based on the measure of movement to determine the estimated position.

8. The method of claim 1, wherein the distorted MR image was acquired with a pulse sequence described by pulse sequence parameters, and wherein the step of generating the simulated MR image is further based on the pulse sequence parameters.

9. The method of claim 8, wherein the step of generating the simulated MR image comprises at least one of:
determining a magnetic field distortion map based on the magnetic susceptibility map and determining a spatial distortion map based on the magnetic field distortion map and the pulse sequence parameters;
determining a magnetic field distortion map based on the magnetic susceptibility map using a dipole field convolution method and determining a spatial distortion map based on the magnetic field distortion map and the pulse sequence parameters; or
determining a magnetic field distortion map based on the magnetic susceptibility map and generating the simulated MR image using a Bloch equation simulation based on based on the magnetic field distortion map and the pulse sequence parameters.

10. The method of claim 9, wherein determining a magnetic field distortion map comprises convolving the set of spatially differentiable functions with a Green's function that relates the magnetic susceptibility map to magnetic field distortions.

11. The method of claim 1, wherein the step of generating the simulated MR image comprises at least one of:
modifying a signal amplitude in the simulated MR image based on a signal loss caused by intravoxel phase dispersion calculated based on the magnetic susceptibility map; or
down-sampling to match a resolution of the simulated MR image with a resolution of the distorted MR image.

12. The method of claim 1, wherein the step of updating the estimated position is performed in k-space.

13. The method of claim 1, wherein the step of updating the estimated position comprises at least one of:
comparing the simulated MR image with the distorted MR image;
generating an objective function that describes an error between the distorted MR image and the simulated MR image, calculating derivatives of the objective function, and reducing the objective function using the derivatives; or
back-propagating gradients and updating the estimated position using a limited-memory Broyden-Fletcher-Goldfarb-Shanno algorithm.

14. The method of claim 13, wherein the objective function comprises a regularization term that discourages at least one of a large deformation of the interventional device; or a large deviation from a known previous position of the interventional device.

15. A method for determining a position of an interventional device arranged in a subject during a magnetic resonance imaging (MRI) acquisition, the method comprising using a processor to perform steps comprising:
accessing a reference image that represents an undistorted MR image of a subject without the interventional device arranged in the subject;
accessing a distorted MR image of the subject with the interventional device arranged in the subject acquired using a pulse sequence;
accessing parameters of the pulse sequence;
estimating a position of the interventional device based on the distorted MR image of the subject;
determining a magnetic field distortion map based on a magnetic susceptibility distribution of the interventional device and the estimated position of the interventional device;
modifying the reference image based on the magnetic field distortion map to produce a simulated image; and
updating the estimated position of the interventional device by comparing the simulated image with the distorted MR image to determine a position of the interventional device arranged in the subject.

16. The method of claim 15, wherein the estimated position of the interventional device is described by at least one of:
a set of spatially differentiable functions; or
a set of Gaussian functions.

17. The method of claim 15, wherein the processor is further configured to at least one of display the position of the interventional device with respect to the subject to a user or control robotic movement of the interventional device based on the position of the interventional device.

18. The method of claim 15, wherein the interventional device is at least one of a needle, a biopsy clip, a deep brain stimulation lead, an electrode lead, or a fiducial marker that has a susceptibility different than a susceptibility of biological tissue.

19. The method of claim 15, wherein the position includes a linear position and an orientation.

20. The method of claim 19, wherein the position further includes a description of a deformation of the interventional device.

21. The method of claim 15, wherein the simulated image is complex-valued, and the distorted MR image is complex-valued.

22. The method of claim 15, wherein the simulated image is a magnitude image, and the distorted MR image is a magnitude image.

23. A system for generating a magnetic resonance (MR) image in the presence of an interventional device that induces distortions, the system comprising a processor configured to perform steps comprising:
accessing a distorted MR image of a subject with the interventional device arranged in the subject;
determining, using the distorted MR image, parameters of a set of spatially differentiable functions describing an estimated position of the interventional device in the subject;
determining a magnetic susceptibility map based on susceptibility properties of the interventional device and the estimated position of the interventional device;
generating a simulated MR image by modifying a reference image of the subject based on the magnetic susceptibility map; and
updating the estimated position using the simulated MR image to generate an updated position of the interventional device in the subject.

24. The system of claim 23, wherein the set of spatially differentiable functions comprises Gaussian functions.

25. The system of claim 23, wherein the position includes at least one of a linear position, an orientation, or a description of a deformation of the interventional device.

26. The system of claim 23, further comprising at least one of:
a user display configured to display the estimated position of the interventional device with respect to the subject; or
a robotic controller configured to control robotic movement of the interventional device based on the estimated position of the interventional device.

27. A method for determining a position of an interventional device arranged in a subject during a magnetic resonance imaging (MRI) acquisition, the method comprising using a processor to perform steps comprising:
accessing a distorted MR field map measured while the interventional device is arranged in the subject acquired using a pulse sequence;
estimating a position of the interventional device based on the distorted MR field map of the subject;
simulating a magnetic field distortion map based on a magnetic susceptibility distribution of the interventional device and the estimated position of the interventional device;
updating the estimated position of the interventional device by comparing the simulated magnetic field distortion map with the distorted MR field map to determine a position of the interventional device arranged in the subject.
